(19) United States
(12) Patent Application Publication
(10) Pub. No.: US 2009/0110639 A1
(43) Pub. Date: Apr. 30, 2009

(54) PREDICTING VACCINE EFFICACY

(75) Inventors: Joseph Monforte, Kensington, CA (US); Jean D. Boyer, (US); David B. Werner, (US)

Correspondence Address: COOLEY GODWARD KRONISH LLP;ATTN: Patent Group, Suite 1100, 777 - 6th Street, NW, WASHINGTON, DC, 20001, US

(73) Assignee: ALTHEA TECHNOLOGIES, INC., San Diego, CA (US)

(21) Appl. No.: 12/136,043

(22) Filed: Jun. 9, 2008

Related U.S. Application Data
(60) Provisional application No. 60/942,658, filed on Jun. 7, 2007.

(51) Int. Cl. A61K 49/00; C07H 21/04; C12Q 1/68; A61P 37/04

(52) U.S. Cl. 424/9.2; 536/23.1; 435/6

(57) ABSTRACT

The present invention provides methods for evaluating a vaccine, combinations of tests and reaction mixtures that can be used as part of such methods, and collections of results comprising results obtained from such methods. The methods, combinations, and collections are useful for determining whether a vaccine is effective and, for example, for comparing the efficacy of different vaccines.

### FIELD OF THE INVENTION

This invention relates generally to vaccines and, more specifically, to methods of predicting whether a vaccine will be effective.

### BACKGROUND OF THE INVENTION

Cellular immune responses play an important role in controlling certain diseases, particularly infectious diseases caused by viral agents (e.g., Human Immunodeficiency Virus (HIV), Hepatitis C Virus (HCV), Influenza, etc.). In addition, cellular immune responses can be important in controlling cell proliferation diseases, such as cancer. Accordingly, vaccines that induce cellular immunity and thereby protect against infectious diseases and proliferative diseases are important.

Suppression of diseases caused by infectious agents or cancer cells typically involves complex cellular immune responses. For example, in the case of Acquired Immune Deficiency Syndrome (AIDS), complex cellular immune responses are associated with long-term non-progression of the disease. Thus, in order for a vaccine to be effective in treating certain infectious diseases or proliferative diseases, it needs to be able to induce complex immune responses. To date, however, there has been only a limited understanding of complex cellular immune responses, and how they can be induced using vaccines. A better understanding of complex cellular immune responses is, thus, central to the design and selection of vaccines that can be used to prevent certain types of infectious diseases and proliferative diseases.

### SUMMARY OF THE INVENTION

The present invention is based, in part, on the discovery that vaccines that prevent the progression of an infection, such as an HIV infection, alter the activity and/or expression profile of immunological genes in a manner that reflects the efficacy of the vaccine. Accordingly, in one aspect, the invention provides methods for evaluating a vaccine in a subject. The methods can include determining, in a vaccinated subject, the activity of two or more immunological genes. The immunological genes can, for example, be selected from the group consisting of genes associated with a pro-inflammatory state, the group consisting of genes associated with induction of a cellular immune response, the group consisting of genes associated with an infection response, or a combination thereof. Alternatively, the method can include determining, in a vaccinated subject, (i) the activity of one or more immunological genes and (ii) a T-cell proliferation rate.

The activity of a particular immunological gene can be measured as a change in activity following ex vivo antigen stimulation of immunological cells, such as peripheral blood mononuclear cells (PBMCs). The change can be an increase or decrease in activity, or there can be no change in activity. For example, following vaccination, the activity of a gene associated with a proinflammatory state can decrease in response to ex vivo stimulation of PBMCs with antigen. Similarly, following vaccination, the activity of a gene associated with the induction of a cellular immune response can increased in response to ex vivo stimulation of PBMCs with antigen. A change in activity following ex vivo antigen stimulation of immunological cells from a vaccinated subject can be compared to an analogous change in activity following ex vivo antigen stimulation of immunological cells from a naïve subject. The comparison can involve subtraction, thereby giving rise to a measurement that is the difference between two measurements of change in activity.

Likewise, the rate of T-cell proliferation can be measured as a change in proliferation rate following ex vivo antigen stimulation of immunological cells, such as peripheral blood mononuclear cells (PBMCs). The change can be an increase or decrease in rate of proliferation, or there can be no change in rate of proliferation. For example, following vaccination, the rate of T-cell proliferation can increase in response to ex vivo stimulation of PBMCs with antigen. A change in rate of T-cell proliferation following ex vivo antigen stimulation of immunological cells from a vaccinated subject can be compared to an analogous change in rate of T-cell proliferation following ex vivo antigen stimulation of immunological cells from a naïve subject. The comparison can involve subtraction, thereby giving rise to a measurement that is the difference between two measurements of change in rate of T-cell proliferation.

The collective result of the activity of at least two immunological genes can be indicative of the efficacy of a vaccine. For example, the collective result of a decrease in the activity of a gene associated with a proinflammatory state and an increase in the activity of a gene associated with the induction of a cellular immune response can be indicative of the efficacy of a vaccine. Similarly, the collective result of the activity of at least one immunological gene in combination with the rate of T-cell proliferation can be indicative of the efficacy of the vaccine. For example, the collective result of a decrease in the activity of a gene associated with a proinflammatory state and/or an increase in the activity of a gene associated with the induction of a cellular immune response, combined with an increase in the rate of T-cell proliferation can be indicative of the efficacy of a vaccine.

The subject can be an animal, such as a bird, a mammal, a primate, or a human. The methods can further comprise administering a vaccine to the subject prior to determining in the subject the activity of two or more immunological genes. In addition, or in the alternative, the methods can further comprise obtaining a sample from a vaccinated subject and determining the activity of one or more immunological genes in the sample from the subject. The sample can be, for example, a blood sample or a sample enriched for PBMCs. Alternatively, the sample can be a tissue sample, such as a tissue biopsy.

The activity of an immunological gene can involve determining the gene's transcription level, translation level, protein activation level, or a combination thereof.

In another aspect, the invention provides combinations of tests useful for predicting whether a vaccine is effective. A combination of tests can include, for example, a first test for the activity of a first gene combined with a second test. The first gene can be, for example, an immunological gene. The first test can involve measuring a change in gene activity following ex vivo antigen stimulation of immunological cells, such as PBMCs. The second test can be a test for the activity of a second gene, such as an immunological gene, and the second test can involve measuring a change in gene activity following ex vivo antigen stimulation of immunological cells, such as PBMCs. Alternatively, the second test can involve measuring the rate of T-cell proliferation, and the second test can involve measuring a change in rate of T-cell proliferation following ex vivo antigen stimulation of immunological cells, such as PBMCs.

A combination of tests can further include three or more tests. Thus, for example, the combination can include a third test for the activity of a third (or second) gene, a fourth test for the activity of a fourth (or third) gene, a fifth test for the activity of a fifth (or fourth) gene, etc. The third, fourth, and/or fifth genes can be, for example, immunological genes. The third, fourth, and/or fifth test can involve, for example, measuring a change in gene activity following ex vivo antigen stimulation of immunological cells, such as PBMCs.

Tests for gene activity can involve, for example, performing PCR. Tests for gene activity can be performed separately, in parallel, or together, such as in a multi-plex PCR reaction.

In another aspect, the invention provides methods for providing useful information for evaluating whether a vaccine is effective. The methods can include determining the activity of a first set of genes, optionally measuring a rate of T-cell proliferation, and providing the activity of the first set of genes and, as appropriate, the rate of T-cell proliferation, to an entity that analyzes the information and provides an evaluation of the vaccine. The first set of genes can, for example, comprise immunological genes. The activity of the first set of genes and, as appropriate, the rate of T-cell proliferation, can be provided in electronic format, a format compatible with a computer algorithm, or a printed format. The first set of genes can include one, two, three, four, five, ten, twenty, fifty, one hundred, or more genes.

In another aspect, the invention provides a collection of results useful for evaluating whether a vaccine is effective. The collection of results can include the values for the activities of a first set of genes and, optionally, a value for a rate of T-cell proliferation. The first set of genes can, for example, comprise immunological genes. The collection of results can be in electronic format, a format compatible with a computer algorithm, or a printed format. The first set of genes can include one, two, three, four, five, ten, twenty, fifty, one hundred, or more genes.

In another aspect, the invention provides a collection of two or more oligonucleotides. The oligonucleotides can be used to determine the activity of a set of genes, such as immunological genes. Individual oligonucleotides can be designed to be used in PCR or as a probe, such as a probe on a microchip. Individual oligonucleotides can be species specific or, in the alternative, can be used to determine the activity of gene homologs present in different species, such as different mammal species (e.g., mice, rates, dogs, cats, primates, and humans).

In another aspect, the invention provides reaction mixtures. The reaction mixtures can include primers or probes useful for determining the activity of a set of genes. The set of genes can, for example, comprise immunological genes. The set of genes can include two, three, four, five, ten, twenty, fifty, one hundred, or more genes. The reaction mixture can further include amplified products, wherein the amplified products correspond to the genes in the set.

### BRIEF DESCRIPTION OF THE DRAWINGS

FIG. 1. IFN-γ-producing cells following the primary immunizations with the SIV gag DNA vaccine and following a rest period. Samples were taken 2 weeks post each injection. PBMCs were isolated by a standard percoll separation technique and assessed for a gag antigen specific response by ELISPOT. The number of cells able to secrete INF-g following SIVgag in vitro stimulation of PBMCs isolated from A) naïve macaques, B) pCSIVgag immunized macaques, and C) pCSIVgag+pmacIL15 immunized macaques is presented as SFC per 1 million PBMCs.

FIG. 2. IFN-γ-producing cells following the primary and secondary set of immunization. Samples were taken two weeks post each injection and assessed for an SIVgag-antigen specific response by ELISpot. The number of cells able to secrete IFN-γfollowing SIVgag in vitro stimulation of PBMCs isolated from A) naïve macaques, B) pCSIVgag, C) pCSIVgag and pmacIL15 immunized macaques is presented as SFC per 1 million PBMCs. D) After the final immunization, the contribution of the CD8 T cells to the observed population of cells from macaques secreting IFN-γwas evaluated.

FIG. 3. Viral load following challenge of Cynomologous macaques with 100 MID of SHIV89.6p. Viral load is presented for A) control, B) SIV DNA, C) SIV DNA+pmacIL15-immunized macaques. The assay has a threshold sensitivity of 200 RNA copies/ml of plasma with interassay variations averaging 0.5 log10. Panel D illustrates the viral loads for the first 15 weeks for each individual macaque. Several animals were sacrificed due to AIDS-like syndrome.

FIG. 4. IFN-γ-producing cells following IV challenge with SHIV89.6p virus. Samples were taken 12 weeks post challenge. PBMCs were isolated by a standard percoll separation technique and assessed for a gag antigen specific response by ELISpot. The number of cells able to secrete IFN-γfollowing SIVgag in vitro stimulation of PBMCs isolated from vaccine naïve macaques, pCSIVgag vaccinated macaques, and pCSIVgag and pmacIL15-vaccinated macaques.

FIG. 5. PD-1 expression in uninfected and infected macaques. PBMCs were stained for CD3, CD4, CD8, and PD-1 expression and analyzed by flow cytometry. Data is presented as the quantification of PD-1 fluorescence on CD3⁺CD4⁺ T cells. (A, C) and CD3⁺CD8⁺ T cells (B, D) of infected vaccinated and infected unvaccinated cynomologous macaques.

FIG. 6. T-cell proliferative responses to SIVgag. PBMC were stained with CFSE and stimulated with SIVgag peptides for 5 days. Standard surface staining protocol was followed for CD3/CD4 positive cells. The data were analyzed using the FlowJo program.

FIG. 7. Gene expression following SIV gag stimulation, quantitative RT-PCR. Five million PBMCs were taken from the macaques 4 weeks post the sixth and final immunization stimulated in vitro with SIVgag peptides and mRNA was extracted. Data is presented for IFN-γ and STAT1.

FIG. 8. Gene expression following SIV gag stimulation, quantitative RT-PCR. Five million PBMCs were taken from the macaques 4 weeks post the sixth and final immunization stimulated in vitro with SIVgag peptides and mRNA was extracted. Data is presented for MMP-9 and IL-8.

FIG. 9. Gene expression following SIV gag stimulation, quantitative RT-PCR. Five million PBMCs were taken from the macaques 4 weeks post the sixth and final immunization stimulated in vitro with SIVgag peptides and mRNA was extracted. Data is presented for other immunological markers of T cell activation.

### DETAILED DESCRIPTION OF THE INVENTION

The present invention provides methods for evaluating a vaccine, combinations of tests, collection of oligonucleotides, and reaction mixtures that can be used as part of such methods, and collections of results comprising experimental values obtained from such methods. The methods, combinations, and collections are useful for determining whether a vaccine is effective and, for example, for comparing the efficacy of different vaccines.

Accordingly, in one aspect, the invention provides methods for evaluating a vaccine in a subject. In certain embodiments, the methods comprise determining, in the subject, the activity of one or more (e.g., 2, 3, 4, 5, 6, 7, 8, 9, 10, 15, 20, 25, 30, 40, 50, 75, 100, or more) immunological genes. In certain embodiments, at least one (e.g., 1, 2, 3, 4, 5, 10, 20, 30, 40, 50, or more) of the immunological genes is selected from the group consisting of genes associated with a proinflammatory state. In other embodiments, at least one (e.g., 1, 2, 3, 4, 5, 10, 20, 30, 40, 50, or more) of the immunological genes is selected from the group consisting of genes associated with induction of a cellular immune response. In other embodiments, at least one (e.g., 1, 2, 3, 4, 5, 10, 20, 30, 40, 50, or more) of the immunological genes is selected from the group consisting of genes associated with infection response. In other embodiments, at least two of the immunological genes are selected from the group consisting of genes associated with a proinflammatory state and genes associated with the induction of a cellular immune response. In still other embodiments, at least one of the immunological genes is selected from the group consisting of genes associated with a proinflammatory state and at least one of the immunological genes is selected from the group consisting of genes associated with induction of a cellular immune response.

As used herein, an “immunological gene” refers to any gene, or protein encoded by the gene, that is associated with an immune response. An immune response is a sequence of events involving a subject's immune system which are triggered by a foreign agent, such as an infectious agent (e.g., a virus, a parasite, a bacterial or fungal cell, a prion, etc.), or by an endogenous agent that is detected by the immune system as a non-self antigen, such as a cancer cell-specific antigen. A gene is “associated with” an immune response if it is involved in stimulating the response, suppressing the response, and/or its activity is modulated as a result of the response. Immunological genes include, but are not limited to, genes associated with a proinflammatory state, genes associated with a cellular immune response, and genes associated with infection response. For example, immunological genes include the genes listed in Table 1. Thus, in certain embodiments, the methods for evaluating a vaccine comprise determining, in the subject, the activity of at least two genes selected from the list of genes shown in Table 1.

As used herein, a “gene associated with a proinflammatory state” is any gene, or protein encoded by the gene, that acts to promote inflammation in a subject or is regulated (e.g., transcriptionally, translationally, and/or in terms of protein activity) in response to inflammation in a subject. The regulation can be up (e.g., increased transcription, increased translation, and/or increased protein activity) or down (e.g., decreased transcription, decreased translation, and/or decreased activity). Genes associated with a proinflammatory state include, but are not limited to, IL-8, MMP-9, and the genes listed in Group 2 of Table 1. Thus, in certain embodiments, the methods for evaluating a vaccine comprise determining, in the subject, the activity of at least two genes selected from the group consisting of the Group 2 genes of Table 1. In other embodiments, the methods for evaluating a vaccine comprise determining, in the subject, the activity of at least one gene selected from the group consisting of the Group 2 genes of Table 1, and at least one other gene selected from the group consisting of the genes of Table 1.

As used herein, a “gene associated with a cellular immune response” is any gene, or protein encoded by the gene, that acts to promote a cellular immune response in a subject or is regulated (e.g., transcriptionally, translationally, and/or in terms of protein activity) in response to a cellular immune response in a subject. A cellular immune response is a T-cell based immune response. The regulation can be up (e.g., increased transcription, increased translation, and/or increased protein activity) or down (e.g., decreased transcription, decreased translation, and/or decreased activity). Genes associated with a cellular immune response include, but are not limited to, IFN-γ, STAT1, IL-10, CD11b, NF-kb, IL-12, IRF-1, and the genes listed in Group 1 of Table 1. Thus, in certain embodiments, the methods for evaluating a vaccine comprise determining, in the subject, the activity of at least two genes selected from the group consisting of the Group 1 genes of Table 1. In other embodiments, the methods for evaluating a vaccine comprise determining, in the subject, the activity of at least one gene selected from the group consisting of the Group 1 genes of Table 1, and at least one other gene selected from the group consisting of the genes of Table 1.

As used herein, a “gene associated with infection response” is any gene, or protein encoded by the gene, that acts to promote a response to infection, whether the response is B-cell mediated, T-cell mediated, or both, or is regulated (e.g., transcriptionally, translationally, and/or in terms of protein activity) as a result of a response to infection. The response can be to any type of infection, such as a viral infection, a parasitic infection, a bacterial infection, a fungal infection, a prion-based infection, etc. The regulation can be up (e.g., increased transcription, increased translation, and/or increased protein activity) or down (e.g., decreased transcription, decreased translation, and/or decreased activity). Genes associated with an infection response include, but are not limited to, the genes listed in Group 3 of Table 1. Thus, in certain embodiments, the methods for evaluating a vaccine comprise determining, in the subject, the activity of at least two genes selected from the group consisting of the Group 3 genes of Table 1. In other embodiments, the methods for evaluating a vaccine comprise determining, in the subject, the activity of at least one gene selected from the group consisting of the Group 3 genes of Table 1, and at least one other gene selected from the group consisting of the genes of Table 1.

In certain embodiments, the methods for evaluating a vaccine comprise determining, in the subject, the activity of at least two genes selected from the group consisting of the Group 4 genes of Table 1. In other embodiments, the methods for evaluating a vaccine comprise determining, in the subject, the activity of at least one gene selected from the group consisting of the Group 4 genes of Table 1, and at least one other immunological gene (e.g., one other gene selected from the group consisting of the genes of Table 1).

In certain embodiments, the methods for evaluating a vaccine comprise determining, in the subject, the activity of at least two genes selected from the group consisting of the Group 5 genes of Table 1. In other embodiments, the methods for evaluating a vaccine comprise determining, in the subject, the activity of at least one gene selected from the group consisting of the Group 5 genes of Table 1, and at least one other immunological gene (e.g., one other gene selected from the group consisting of the genes of Table 1).

In certain embodiments, the methods for evaluating a vaccine comprise determining, in the subject, the activity of at least one gene selected from the group consisting of INF-γ and STAT1, and at least one other immunological gene. In certain embodiments, the methods for evaluating a vaccine comprise determining, in the subject, the activity of at least one gene selected from the group consisting of MMP-9 and IL-8, and at least one other immunological gene. In other embodiments, the methods for evaluating a vaccine comprise determining, in the subject, the activity of at least one gene selected from the group consisting of INF-γ and STAT1, and at least one gene selected from the group consisting of MMP-9 and IL-8.

As used herein, the “activity” of a gene refers to a measure of the amount of gene expression, gene product (i.e., protein), or activated gene product (i.e., activated protein) present in a sample. The activity of a gene can be determined, for example, based on the gene transcription level (i.e., the amount of RNA, such as mRNA), the gene translation level (i.e., the amount of protein product), the protein activation level (which can depend, e.g., on postranslational modifications, such as phosphorylation or changes in subcellular localization), or a combination thereof.

In certain embodiments, the activity of a gene is determined from the gene's transcription level. For example, in certain embodiments, the activity of a gene is determined by performing PCR, e.g., multiplex PCR, on RNA isolated from a subject (e.g., a sample from a subject, such as blood cells or PBMCs). Methods for performing PCR that are suitable for use in the methods of the invention have been described, for example, in U.S. Pat. No. 6,618,679. In preferred embodiments, performing PCR provides quantitative information about a gene's expression level. In other embodiments, the activity of a gene is determined using DNA microchips to quantify the amount of gene expression. In still other embodiments, the activity of a gene is determined by electrophoretically separating RNA isolated from a sample on a gel (e.g., a polyacrylamide gel) and using a probe (e.g., a fluorescently labeled or radioactive probe) to quantify that amount of gene expression. In certain embodiments, a gene's transcription level is determined with respect to a standard, such as an internal standard (e.g., the expression level of a beta-actin (ACTB) gene, a glyceraldehyde-3-phosphate dehydrogenase (GAPD) gene, a cyclophilin A (cycloA) gene, or some other gene that is not upregulated or downregulated when a subject's immune system responds to an infectious agent). The methods of quantifying a gene's transcription level disclosed herein are not intended to be limiting. Persons skilled in the art will understand that there are many different techniques that can be employed to determine a gene's expression level, any of which could be used in the methods of the present invention.

In certain embodiments, the activity of a gene is determined based on the gene's translation level (i.e., the level of protein product). In certain embodiments, a gene's translation level is determined using an immunological assay, such as an ELISA, an immunoprecipitation experiment, a western blot, FACS analysis (e.g., for cell surface proteins), etc. Immunological assays can involve the use of any number of different types of antibodies, depending upon the specific assay and the protein product to be detected. In other embodiments, a gene's translation level is determined using a binding assay (e.g., involving a target protein or other ligand that binds specifically to the protein product of the gene being assayed) or an enzymatic assay. In still other embodiments, a gene's translation level is determined using mass spectrometry, such as LC/MS/MS. The methods of quantifying a gene's translation level disclosed herein are not intended to be limiting. Persons skilled in the art will understand that there are many different techniques that can be employed to determine a gene's translation level, any of which could be used in the methods of the present invention.

In certain embodiments, the activity of a gene is determined based on the gene's protein activation level (i.e., the level of activated protein product). In certain embodiments, a gene's protein activation level is determined using an immunological assay, such as an ELISpot assay, an immunoprecipitation experiment, a western blot, FACS analysis (e.g., for cell surface proteins), etc. Immunological assays can involve the use of any number of different types of antibodies, depending upon the specific assay and the protein product to be detected. In other embodiments, a gene's protein activation level is determined using a binding assay (e.g., involving a target protein or other ligand that binds specifically to activated protein) or an enzymatic assay. The methods of quantifying a gene's protein activation level disclosed herein are not intended to be limiting. Persons skilled in the art will understand that there are many different techniques that can be employed to determine a gene's protein activation level, any of which could be used in the methods of the present invention.

In certain embodiments, the activity of a gene corresponds to the gene's transcription level. In other embodiments, the activity of a gene corresponds to the gene's translation level. In still other embodiments, the activity of a gene corresponds to the gene's protein activation level.

In certain embodiments, the activity of a gene in a vaccinated subject is measured relative to the activity of the corresponding gene in an unvaccinated (i.e., naive) subject. For example, the activity of a gene in an unvaccinated subject (or the average activity of a gene in a group of unvaccinated subjects) can be subtracted from the activity of a gene in a vaccinated subject. Thus, the activity of a gene in a vaccinated subject can be increased, the same as, or decreased relative to the activity of the corresponding gene in an unvaccinated subject or group of unvaccinated subjects.

In other embodiments, the activity of a gene in a vaccinated subject is measured relative to the activity of the corresponding gene is an infected subject (e.g., a subject that is infected with a particular infectious agent, such as HIV) or a subject that has a proliferative disease (e.g., cancer). In certain embodiments, the activity of a gene in a vaccinated subject is measured relative to the average activity of the corresponding gene in a group of infected subjects or a group of subjects that have a proliferative disease. Thus, the activity of a gene in a vaccinated subject can be increased, the same as, or decreased relative to the activity of the corresponding gene in an infected subject, a subject that has a proliferative disease, or group of infected subjects or subjects that have a proliferative disease.

In certain embodiments, the activity of a gene in a vaccinated subject is measured as a change in activity following ex vivo antigen stimulation of immunological cells, such as blood cells of PBMCs, from the subject. The activity of a gene in ex vivo antigen stimulated immunological cells from a vaccinated subject can be increased, the same as, or decreased relative to the activity of the same gene in immunological cells that have not been stimulated with antigen ex vivo. In certain embodiments, the activity of a gene in a vaccinated subject is measured as a change between vaccinated and unvaccinated subjects of a change in activity following ex vivo antigen stimulation. For example, a change in activity following ex vivo antigen stimulation of immunological cells can be determined for both a vaccinated subject and an unvaccinated subject, and the change measured for the unvaccinated subject can be subtracted from the change measured for the vaccinated subject.

In certain embodiments, the methods for evaluating a vaccine further comprise evaluating the proliferation of a lymphocyte cell population in a subject. A lymphocyte cell population can be, e.g., a T-cell population or a B-cell population. In certain embodiments, the lymphocyte population is a T-cell population, such as a CD4+ T-cell population, a CD8+ T-cell population, or a combination of CD4+ and CD8+ T-cells. In certain embodiments, evaluating the proliferation of a lymphocyte cell population comprises treating the lymphocyte cell population with a fluorescent dye (e.g., CFSE) and, at a time thereafter, analyzing the treated cells using FACS.

In certain embodiments, the proliferation of a lymphocyte population comprises ex vivo antigen stimulation of the lymphocyte population prior to evaluating proliferation. In such embodiments, the measure of lymphocyte proliferation can be a change in the amount of proliferation occurring in lymphocytes that have been stimulated with antigen, as compared to lymphocytes that have not been stimulated with antigen. In related embodiments, the measure of lymphocyte proliferation can further involve comparing a change in lymphocyte proliferation upon antigen stimulation in lymphocytes from vaccinated and unvaccinated subjects (e.g., the change in lymphocyte proliferation observed in lymphocytes from unvaccinated subjects can be subtracted from the change in lymphocyte proliferation observed in vaccinated subjects).

In certain embodiments, a collective result of the activity of at least two immunological genes is indicative of the efficacy of the vaccine. In certain embodiments, the at least two immunological genes comprise genes associated with a proinflammatory state, genes associated with induction of a cellular immune response, genes associated with an infection response, or any combination thereof. In certain embodiments, the at least two immunological genes comprise genes listed in Table 1.

In certain embodiments, a decrease in the activity of a gene associated with the proinflammatory state is indicative of the efficacy of the vaccine. In certain embodiments, a decrease in the activity of a gene from Group 2 of Table 1 is indicative of the efficacy of the vaccine. In certain embodiments, the decrease in the activity of a gene associated with the proinflammatory state is a decrease relative to an unvaccinated (i.e., naïve) subject. In other embodiments, the decrease in the activity of a gene associated with the proinflammatory state is a decrease relative to an infected subject. In still other embodiments, the decrease in the activity of a gene associated with the proinflammatory state is a decrease in gene activity following ex vivo antigen stimulation of immunological cells, such as blood cells of PBMCs, from the vaccinated subject. In certain embodiments, the decrease in gene activity following ex vivo antigen stimulation of immunological cells from a vaccinated subject is greater than the decrease in gene activity following ex vivo antigen stimulation of immunological cells from a naïve subject. In certain embodiments, a decrease in the activity of Il-8 and/or MMP-9 is indicative of the efficacy of a vaccine.

In certain embodiments, an increase in the activity of a gene associated with a cellular immune response is indicative of the efficacy of the vaccine. In certain embodiments, an increase in the activity of a gene from Group 1 of Table 1 is indicative of the efficacy of the vaccine. In certain embodiments, the increase in the activity of a gene associated with a cellular immune response is an increase relative to an unvaccinated (i.e., naïve) subject. In other embodiments, the increase in the activity of a gene associated with a cellular immune response is an increase in gene activity following ex vivo antigen stimulation of immunological cells, such as blood cells of PBMCs, from the vaccinated subject. In certain embodiments, the increase in gene activity following ex vivo antigen stimulation of immunological cells from a vaccinated subject is greater than the increase in gene activity following ex vivo antigen stimulation of immunological cells from a naïve subject. In certain embodiments, an increase in the activity of INF-γ and/or STAT1 is indicative of the efficacy of a vaccine. In certain embodiments, a rapid increase in INF-γ (e.g., an increase following one or two vaccinations) is further indicative of the efficacy of the vaccine.

In certain embodiments, a decrease in the activity of a gene associated with the proinflammatory state combined with an increase in the activity of a gene associated with a cellular immune response is indicative of the efficacy of the vaccine. In certain embodiments, a decrease in the activity of a gene from Group 2 of Table 1 combined with an increase in the activity of a gene from Group 1 of Table 1 is indicative of the efficacy of the vaccine. In certain embodiments, a decrease in the activity of a gene selected from the group consisting of MMP-9 and IL-8 combined with an increase in the activity of a gene selected from the group consisting of INF-γ and STAT1 is indicative of the efficacy of the vaccine.

In certain embodiments, a decrease in the activity of a gene associated with the proinflammatory state, combined with an increase in the activity of a gene associated with a cellular immune response, and further combined with an increase in T-cell proliferation is indicative of the efficacy of the vaccine.

In certain embodiments, a decrease in the activity of PD-1 (e.g., PD-1 protein expression levels) is indicative of the efficacy of a vaccine. In certain embodiments, the decrease is a decrease relative to an unvaccinated subject. In certain embodiments, the decrease in the activity of PD-1 is a decrease in gene activity following ex vivo antigen stimulation of immunological cells, such as blood cells of PBMCs, from a vaccinated subject. In certain embodiments, the decrease in gene activity following ex vivo antigen stimulation of immunological cells from a vaccinated subject is greater than the decrease in gene activity following ex vivo antigen stimulation of immunological cells from a naïve subject. In certain embodiments, the decrease PD-1 activity is on CD8+ T-cells. In other embodiments, the decrease in PD-1 activity is on CD4+ T-cells. In still other embodiments, the decrease is on both CD4+ and CD8+ T-cells. In certain embodiments, a decrease in the activity of PD-1, combined with any other increase or decrease in immunological gene activity described herein, is indicative of the efficacy of a vaccine.

In certain embodiments, an increase in T-cell proliferation is indicative of the efficacy of the vaccine. In certain embodiments, the T-cells are CD4+ T-cells, CD8+ T-cells, or both. In certain embodiments, the increase is an increase relative to an unvaccinated subject. In certain embodiments, an increase in T-cell proliferation, combined with any other increase or decrease in immunological gene activity described herein, is indicative of the efficacy of a vaccine.

In certain embodiments, the subject is an animal, such as a bird (e.g., a chicken, duck, etc.), a mammal (e.g., a mouse, rat, guinea pig, rabbit, dog, cat, goat, pig, cow, horse, etc.), a primate (e.g., a macaque monkey, chimpanzee, etc.), or a human (e.g., Homo sapiens, Neanderthal, Cro-Magnon, etc.).

In certain embodiments, the methods further comprise administering a vaccine to a subject prior to determining, in the subject, the activity of two or more immunological genes. As used herein, a “vaccine” is any agent capable of eliciting an immune response. Examples of vaccines include, but are not limited to, DNA vaccines, proteins, peptides, infectious agents (e.g., infectious agents that have been heat inactivated or attenuated), etc. In certain embodiments, vaccines are administered two or more times in order to elicit a detectable immune response. In certain embodiments, the time between administering two vaccinations is relatively short (e.g., 1, 2, 3, 4 weeks or longer). In other embodiments, the time between administering two vaccinations is relatively long (e.g., 6 months, 1 year, 1.5 years, or longer).

In certain embodiments, a vaccine is administered with an adjuvant. Suitable adjuvants include, for example, aluminum salts (e.g., aluminum phosphate, aluminum hydroxide, etc.), organic compounds (e.g., phosphate, squalene, etc.), oil-based adjuvants, and virosomes (e.g., containing a membrane-bound influenza heamaglutinnin and/or neuraminidase proteins). The adjuvants disclosed herein are not intended to be limiting. Persons skilled in the art will understand that there are many different adjuvants that can be employed in combination with vaccines used in the methods of the present invention. In other embodiments, a vaccine is administered in conjunction with a cytokine, such as IL-12, IL-15, etc.

In certain embodiments, the activity of two or more immunological genes is determined in the subject by determining the activity of two or more immunological genes in a sample from the subject. In certain embodiments, the methods further comprise obtaining a sample from the subject. In certain embodiments, the sample is a blood sample. In certain embodiments, the sample is enriched for PBMCs (e.g., CD4+ lymphocytes, CD8+ lymphocytes, or a combination thereof). PBMCs can be obtained using standard isolation procedures, such as centrifugation (e.g., using Becton Dickinson Vacutainer CPT™ Cell Preparation Tubes).

In another aspect, the invention provides methods for comparing the efficacy of two vaccines comprising evaluating a first vaccine in a first subject and evaluating a second vaccine in a second subject, wherein evaluating the first and second vaccines comprises determining the activity of at least two immunological genes in both the first and second subjects, and then comparing the activity measurements determined for the at least two immunological genes. In certain embodiments, comparing the activity measurements determined for the at least two immunological genes comprises creating a differences profile. As used herein, a “differences profile” is a set of values showing the differences between the activity values measured for each of the at least two immunological genes in the first and second subjects. The methods for comparing can comprise any of the methods for evaluating a vaccine disclosed herein.

In certain embodiments, the vaccine that has a greater decrease in the activity of at least one gene associated with a proinflammatory state is a superior vaccine. For example, in certain embodiments, a vaccine that results in a greater decrease in the level of MMP-9 and/or IL-8 is a superior vaccine. In other embodiments, the vaccine that has a larger increase in the activity of at least one gene associated with a cellular immune response is a superior vaccine. For example, in certain embodiments, a vaccine that results in a greater increase in the level of INF-γ and/or STAT1 is a superior vaccine. In still other embodiments, the vaccine that has a larger decrease in the activity of at least one gene associated with a proinflammatory state and a larger increase in the activity of at least one gene associated with a cellular immune response is a superior vaccine. For example, in certain embodiments, a vaccine that results in a greater decrease in the level of MMP-9 and/or IL-8 and a greater increase in the level of INF-γ and/or STAT1 is a superior vaccine.

In another aspect, the invention provides combinations of tests useful for predicting whether a vaccine is effective. In certain embodiments, the combination of tests comprises a first test for the activity of a first gene and a second test, wherein the first gene is an immunological gene (e.g., any gene in Table 1). In certain embodiments, the second test measures the rate of T-cell proliferation. In certain embodiments, the first gene is associated with a proinflammatory state (e.g., MMP-9 or IL-8). In other embodiments, the first gene is associated with a cellular immune response (e.g., INF-γ or STAT1). In still other embodiments, the first gene is PD-1.

In other embodiments, the second test is for the activity of a second gene, wherein the second gene is an immunological gene (e.g., any gene in Table 1). In certain embodiments, the first gene is associated with a proinflammatory state (e.g., MMP-9 or IL-8) and the second gene is a different immunological gene (e.g., another gene from Table 1, such as a gene associated with a cellular immune response). In certain embodiments, the first gene is associated with a cellular immune response (e.g., INF-γ or STAT1), and the second gene is a different immunological gene (e.g., another gene from Table 1, such as a gene associated with a proinflammatory state). In certain embodiments, the first gene is associated with a proinflammatory state and the second gene is associated with a cellular immune response.

In certain embodiments, the combination of tests further comprises a third test. In certain embodiments, the third test is for the activity of a second immunological gene (e.g., a gene from Table 1). In other embodiments, the third test is for the activity of a third immunological gene. In certain embodiments, the combination of tests comprises 4, 5, 6, 7, 8, 9, 10, 15, 20, 30, 40, 50, or more tests. In certain embodiments, each test is for the activity of an immunological gene. In other embodiments, all but one of the tests is for the activity of an immunological gene, and the one other test measures T-cell proliferation.

Each test for measuring gene activity can employ any method disclosed herein for such purpose, as well as other methods for measuring gene activity known the persons skilled in the art. In certain embodiments, the test of gene activity is a test for gene transcription levels. In certain embodiments, the test of gene activity comprises PCR amplification of a sample from a vaccinated subject. In certain embodiments, the tests are performed separately, in parallel, or altogether, e.g., using multiplex PCR.

In another aspect, the invention provides methods for providing useful information for evaluating whether a vaccine is effective. In certain embodiments, the methods comprise determining the activity of a first set of genes and, optionally, a rate of T-cell proliferation, and providing the activity of the first set of genes and, as appropriate, the rate of T-cell proliferation, to an entity that analyzes the information (i.e., activity measurements and, optionally, T-cell proliferation rate) and provides an evaluation of the vaccine. In certain embodiments, the first set of genes comprises immunological genes (e.g., genes associated with a proinflammatory state, genes associated with the induction of a cellular immune response, genes associated with an infection response, or any combination thereof, including any combination disclosed herein). In certain embodiments, the activity of the first set of genes is provided in electronic format or a format compatible with a computer algorithm. In other embodiments, the activity of the first set of genes is provided in a printed format (e.g., hand-written, typed, or printed). In certain embodiments, the first set of genes includes 1, 2, 3, 4, 5, 6, 7, 8, 9, 10, 15, 20, 25, 30, 35, 40, 45, 50, 60, 70, 80, 90, 100, or more genes.

As used herein, an “entity that analyzes the activity” can be an individual, such as a scientist or medical doctor, a business, such as a corporation or partnership, or a governmental agency, such as the National Institutes for Health, the National Science Foundation, or the Center for Disease Control. Each test for measuring gene activity can employ any method disclosed herein for such purpose, as well as other methods for measuring gene activity known the persons skilled in the art.

In another aspect, the invention provides a collection of results useful for evaluating whether a vaccine is effective. In certain embodiments, the collection of results includes the values for the activities of a first set of genes and, optionally, a value for a rate of T-cell proliferation. In certain embodiments, the first set of genes comprises immunological genes. In certain embodiments, the activity of the first set of genes and, if appropriate, the rate of T-cell proliferation, is provided in electronic format or a format compatible with a computer algorithm. In other embodiments, the activity of the first set of genes is provided in a printed format (e.g., hand-written, typed, or printed). In certain embodiments, the first set of genes includes 2, 3, 4, 5, 6, 7, 8, 9, 10, 15, 20, 25, 30, 35, 40, 45, 50, 60, 70, 80, 90, 100, or more genes.

In another aspect, the invention provides a collection of two or more oligonucleotides. In certain embodiments, the collection can be used to determine the activity of a set of immunological genes. In certain embodiments, the collection can be used to determine the activity of a set of immunological genes in two or more different subjects. In certain embodiments, the subjects are from different species (e.g., birds, mice, rats, dogs, cats, primates, humans). In certain embodiments, the collection comprises probes for determining the gene expression level of a set of immunological genes. In other embodiments, the collection comprises probes for determining the gene translation levels of a set of immunological genes (e.g., oligonucleotide probes can be used to determine the activity of immunological genes that directly bind to such probes, such as immunological transcription factors). In certain embodiments, the oligonucleotides are probes that are provided on a solid support, such as a microchip.

In other embodiments, the oligonucleotides are primers that are provided alone or in combination. In certain embodiments, the oligonucleotides are primers that are provided in a dry form (e.g., lyophilized) or an aqueous form (e.g., in water or a buffer).

As used herein, an “oligonucleotide” is a nucleic acid polymer such as DNA, RNA, PNA, or other polymers containing modified nucleic acid bases.

As used herein, a “primer” is a small oligonucleotide (e.g., DNA, RNA, PNA, or other modified nucleic acid molecule) that can be used to amplify (e.g., by PCR) a nucleic acid molecule, such as RNA or DNA. Suitable primers for use in the methods of the invention can include gene-specific primers and, optionally, universal primers, as described in U.S. Pat. No. 6,618,679. Persons skilled in the art will understand that there are many different primers sequences that can be employed to amplify a particular nucleic acid sequence, any of which could be used in the methods of the present invention.

As used herein, a “probe” is a small oligonucleotide (e.g., DNA, RNA, PNA, or other modified nucleic acid molecule) that can be used to hybridize to a target nucleic acid molecule, such as an immunological gene transcript, that is in solution or present on a blot. In many cases, primers and probes can be used interchangeably.

In another aspect, the invention provides reaction mixtures. In certain embodiments, the reaction mixtures comprise primers or probes useful for determining the activity of a set of genes. In certain embodiments, the set of genes comprises immunological genes. In certain embodiments, the first set of genes includes 2, 3, 4, 5, 6, 7, 8, 9, 10, 15, 20, 25, 30, 35, 40, 45, 50, 60, 70, 80, 90, 100, or more genes.

In certain embodiments, the reaction mixture further comprises amplified products, wherein the amplified products correspond to the genes in the set. As used herein, an “amplified product” is a nucleic acid molecule generated by any known means of amplifying nucleic acids, including PCR.

The following examples illustrate the use of the methods of the invention to evaluate vaccine efficacy. The examples should, of course, be understood to be merely illustrative of only certain embodiments of the invention and not to constitute limitations upon the scope of the invention which is defined by the claims that are appended at the end of this description.

### EXAMPLES

### Example 1

Table 1 contains a list of exemplary immunological genes divided into different categories/groups.

TABLE 1

| | | Gene | Gene |
|---|---|---|---|
| | Full Gene Name | Symbol | Accession |
| Group 1: | cytochrome P450, family 1, subfamily B, | CYP1B1 | NM_000104 |
| Cellular | polypeptide 1 (CYP1B1) | | |
| Immune | intercellular adhesion molecule 1 (CD54), | CD54, | NM_000201 |
| Response | human rhinovirus receptor (ICAM1) | ICAM1 | |
| | cytochrome P450, family 1, subfamily A, | CYP1A1 | NM_000499 |
| | polypeptide 1 (CYP1A1) | | |
| | tumor protein p53 (Li-Fraumeni syndrome) | TP53 | NM_000546 |
| | (TP53) | | |
| | interleukin 1, beta (IL1B) | IL1B | NM_000576 |
| | interleukin 15 (IL15), | IL15 | NM_000585 |
| | CD14 antigen (CD14) | CD14 | NM_000591 |
| | lymphotoxin alpha (TNF superfamily, member 1) | LTA | NM_000595 |
| | (LTA) | | |
| | CD4 antigen (p55) (CD4) | p55, CD4 | NM_000616 |
| | nitric oxide synthase 2A (inducible, hepatocytes) | NOS2A | NM_000625 |
| | (NOS2A), | | |
| | colony stimulating factor 2 (granulocyte- | CSF2 | NM_000758 |
| | macrophage) (CSF2) | | |
| | cytochrome P450, family 1, subfamily A, | CYP1A2 | NM_000761 |
| | polypeptide 2 (CYP1A2) | | |
| | prostaglandin-endoperoxide synthase 2 | PTGS2 | NM_000963 |
| | (prostaglandin G/H synthase and | | |
| | cyclooxygenase) (PTGS2) | | |
| | caspase 9, apoptosis-related cysteine peptidase | CASP9 | NM_001229 |
| | (CASP9), | | |
| | CD8 antigen, alpha polypeptide (p32) (CD8A), | CD8A | NM_001768 |
| | v-myc myelocytomatosis viral oncogene | MYC | NM_002467 |
| | homolog (avian) (MYC) | | |
| | protein tyrosine phosphatase, receptor type, C | PTPRC | NM_002838 |
| | (PTPRC), | | |
| | nuclear factor of kappa light polypeptide gene | p105, | NM_003998 |
| | enhancer in B-cells 1 (p105) (NFKB1) | NFkB1 | |
| | caspase 3, apoptosis-related cysteine peptidase | CASP3 | NM_004346 |
| | (CASP3), | | |
| | interleukin 23, alpha subunit p19 (IL23A) | IL23A | NM_016584 |
| | scinderin (SCIN) | SCIN | NM_033128 |
| Group 2: Pro- | HUM4COLA Human type IV collagenase | MMP9 | J05070 |
| Inflammatory | HUMIL1 Human monocyte interleukin 1 (IL-1) | IL1 | K02770 |
| | HUMIFNB2B Human hybridoma growth factor | IL6 | M18403 |
| | (interleukin 6) | | |
| | interleukin 10 (IL10) | IL10 | NM_000572 |
| | interleukin 8 (IL8) | IL8 | NM_000584 |
| | interleukin 2 (IL2) | IL2 | NM_000586 |
| | interleukin 4 (IL4), | IL4 | NM_000589 |
| | tumor necrosis factor (TNF superfamily, member | TNF | NM_000594 |
| | 2) (TNF) | | |
| | interferon, gamma (IFNG) | IFNG | NM_000619 |
| | fibroblast growth factor 1 (acidic) (FGF1), | FGF1 | NM_000800 |
| | interleukin 18 (interferon-gamma-inducing | IL18 | NM_001562 |
| | factor) (IL18) | | |
| | CD63 antigen (melanoma 1 antigen) (CD63) | CD63 | NM_001780 |
| | connective tissue growth factor (CTGF) | CTGF | NM_001901 |
| | interleukin 12B (natural killer cell stimulatory | IL12B | NM_002187 |
| | factor 2, cytotoxic lymphocyte maturation factor | | |
| | 2, p40) (IL12B) | | |
| | chemokine (C-C motif) ligand 1 (CCL1) | CCL1 | NM_002981 |
| | nuclear factor of kappa light polypeptide gene | p105, | NM_003998 |
| | enhancer in B-cells 1 (p105) (NFKB1) | NFkB1 | |
| | signal transducer and activator of | STAT1 | NM_139266 |
| | colony stimulating factor 1 (macrophage) | CSF1 | NM_172210 |
| | (CSF1), | | |
| | membrane cofactor protein (CD46, trophoblast- | CD46, | NM_172350 |
| | lymphocyte cross-reactive antigen) (MCP), | MCP | |
| | HSU03905 Human monocyte chemoattractant | MCP-1RB | U03905 |
| | protein 1 receptor (MCP-1RB) alternatively | | |
| | spliced | | |
| | HSIRF1 Human | IRF-1 | X14454 |
| | HSGPCRIN8 H. sapiens | HSGPCRIN8 | X95876 |
| Group 3: | interleukin 10 (IL10) | IL10 | NM_000572 |
| Infection | interleukin 1, beta (IL1B) | IL1B | NM_000576 |
| Response | interleukin 2 (IL2) | IL2 | NM_000586 |
| | interleukin 6 (interferon, beta 2) (IL6) | IL6 | NM_000600 |
| | transforming growth factor, beta 1 (Camurati- | TGFB1 | NM_000660 |
| | Engelmann disease) (TGFB1) | | |
| | ribosomal protein S9 (RPS9) | RPS9 | NM_001013 |
| | mitogen-activated protein kinase 14 (MAPK14), | MAPK14 | NM_001315 |
| | cyclin D3 (CCND3) | CCND3 | NM_001760 |
| | dipeptidylpeptidase 4 (CD26, adenosine | CD26, | NM_001935 |
| | deaminase complexing protein 2) (DPP4) | DPP4 | |
| | endothelin 1 (EDN1) | EDN1 | NM_001955 |
| | platelet factor 4 (chemokine (C—X—C motif) ligand | PF4 | NM_002619 |
| | 4) (PF4) | | |
| | pro-platelet basic protein (chemokine (C—X—C | PPBP | NM_002704 |
| | motif) ligand 7) (PPBP) | | |
| | S100 calcium binding protein A8 (calgranulin A) | S100A8 | NM_002964 |
| | (S100A8) | | |
| | selectin P ligand (SELPLG) | SELPLG | NM_003006 |
| | toll-like receptor 2 (TLR2) | TLR2 | NM_003264 |
| | tumor necrosis factor receptor superfamily, | TNFRSF14 | NM_003820 |
| | member 14 (herpesvirus entry mediator) | | |
| | (TNFRSF14) | | |
| | S100 calcium binding protein A12 (calgranulin | S100A12 | NM_005621 |
| | C) (S100A12) | | |
| | prominin 1 (PROM1) | PROM1 | NM_006017 |
| | tissue factor pathway inhibitor (lipoprotein- | TFPI | NM_006287 |
| | associated coagulation inhibitor) (TFPI), | | |
| | dual specificity phosphatase 10 (DUSP10), | DUSP10 | NM_007207 |
| | toll-like receptor 4 (TLR4), | TLR4 | NM_138554 |
| | alpha-2-macroglobulin (A2M) | A2M | NM_000014 |
| | adenomatosis polyposis coli (APC) | APC | NM_000038 |
| | Bruton agammaglobulinemia tyrosine kinase | BTK | NM_000061 |
| | (BTK) | | |
| | interleukin 8 receptor, alpha (IL8RA) | IL8RA | NM_000634 |
| | S-adenosylhomocysteine hydrolase (AHCY) | AHCY | NM_000687 |
| | chemokine (C—X—C motif) ligand 1 (melanoma | CXCL1 | NM_001511 |
| | growth stimulating activity, alpha) (CXCL1) | | |
| | CD69 antigen (p60, early T-cell activation | CD69 | NM_001781 |
| | antgien) (CD69) | | |
| | casein kinase 1, delta (CSNK1D), | CSNK1D | NM_001893 |
| | interleukin 6 signal transducer (gp130, | IL6ST | NM_002184 |
| | oncostatin M receptor) (IL6ST), | | |
| | TIMP metallopeptidase inhibitor 1 (TIMP1) | TIMP1 | NM_003254 |
| | endothelial differentiation, G-protein-coupled | EDG6 | NM_003775 |
| | receptor 6 (EDG6) | | |
| | CASP8 and FADD-like apoptosis regulator | CFLAR | NM_003879 |
| | (CFLAR) | | |
| | caspase 5, apoptosis-related cysteine peptidase | CASP5 | NM_004347 |
| | (CASP5) | | |
| | CD74 antigen (invariant polypeptide of major | CD74 | NM_004355 |
| | histocompatibility complex, class II antigen- | | |
| | associated) (CD74), | | |
| | interleukin 16 (lymphocyte chemoattractant | IL16 | NM_004513 |
| | factor) (IL16), | | |
| | integrin, alpha 3 (antigen CD49C, alpha 3 | ITGA3 | NM_005501 |
| | subunit of VLA-3 receptor) (ITGA3), | | |
| | scavenger receptor class B, member 1 | SCARB1 | NM_005505 |
| | (SCARB1) | | |
| | activating | ATF6 | NM_007348 |
| | BRF2, subunit of RNA polymerase III | BRF2 | NM_018310 |
| | signal transducer and activator of | STAT3 | NM_139276 |
| | chemokine (C-C motif) ligand 28 (CCL28), | CCL28 | NM_148672 |
| | phosphoinositide-3-kinase, regulatory subunit 1 | PIK3R1 | NM_181523 |
| | (p85 alpha) (PIK3R1), | | |
| | TSC22 domain family, member 3 (TSC22D3), | TSC22D3 | NM_198057 |
| Group 4 | integrin, alpha M (complement component 3 | CD11b | NM_000632 |
| | receptor 3 subunit) (ITGAM) | | |
| | interleukin 6 (interferon, beta 2) (IL6) | IL6 | NM_000600 |
| | transforming growth factor beta 1 (Camurati- | TGFb | NM_000660 |
| | Engelmann disease) (TGFB) | | |
| | interleukin 8 (IL8) | IL8 | NM_000584 |
| | HSIRF1 Human | IRF-1 | NM_002198 |
| | interleukin 10 (IL10) | IL10 | NM_000572 |
| | interleukin 4 (IL4) | IL4 | NM_000589 |
| | colony stimulating factor 1 (macrophage) (CSF1) | CSF | NM_172210 |
| | chemokine (C-C motif) receptor 2 (CCR2) | CCR2 | NM_000648/ |
| | | | 647 |
| | HUM4COLA Human type IV collagenase | MMP9 | NM_004994 |
| | granzyme A (granzyme 1, cytotoxic T- | GZMA | NM_006144 |
| | lymphocyte-associated serine esterase 3) | | |
| | (GZMA) | | |
| | granzyme B (granzyme 2, cytotoxic T- | GZMB | NM_004131 |
| | lymphocyte-associated serine esterase 1) | | |
| | (GZMB) | | |
| | nuclear factor of kappa light polypeptide gene | p105, | NM_003998 |
| | enhancer in B-cells 1 (p105) (NFKB1) | NFkB1 | |
| | tumor necrosis factor, alpha (TNF) | TNFalpha | NM_000594 |
| | signal transducer and activator of | STAT1 | NM_139266 |
| | fibroblast growth factor 1 (acidic) (FGF1) | FGF | NM_000800 |
| | programmed cell death 1 (PDCD1) | PD1 | NM_005018 |
| | chemokine (C-C motif) ligand 4 (CCL4) | CCL4 | NM_002984 |
| | perforin 1 (pore forming protein) (PRF1) | Perforin | NM_005041 |
| | glyceraldehyde-3-phosphate dehydrogenase | GAPDH | NM_002046 |
| | (GAPDH) | | |
| | actin, beta (ACTB) | actin | NM_001101 |
| | CD46 molecule, complement regulatory protein | MCP1 | NM_172350 |
| | (CD46) | | |
| | interleukin 18, interferon-gamma-inducing factor | IL18 | NM_001562 |
| | (IL18) | | |
| | chemokine (C—X—C motif) receptor 3 (CXCR3) | CXCR3 | NM_001504 |
| | interferon gamma (IFNG) | IFNG | NM_000619 |
| Group 5 | antigen identified by monoclonal antibody Ki-67 | KI67 | NM_002417 |
| | (MKI67) | | |
| | Homo sapiens cytotoxic T-lymphocyte- | CTLA4 | NM_005214 |
| | associated protein 4 (CTLA4) | | |
| | interferon, gamma (IFNG) | IFNG | NM_000619 |
| | signal transducer and activator of transcription | STAT1 | NM_139266 |
| | 1, 91 kDa (STAT1) | | |
| | T-box 21 (TBX21) | TBET | NM_013351 |
| | eomesodermin homolog (Xenopus laevis) | EOMES | NM_005442 |
| | (EOMES) | | |
| | programmed cell death 1 (PDCD1) | PD1 | NM_005018 |
| | PD-1 Ligand | PD1L | NM_021893 |
| | phospholipase C, beta 1 (phosphoinositide- | MIP-1beta | NM_015192 |
| | specific) (PLCB1) | | |
| | chemokine (C-C motif) ligand 5 (CCL5) | RANTES | NM_002985 |
| | granzyme B (granzyme 2, cytotoxic T- | GZMB | NM_004131 |
| | lymphocyte-associated serine esterase 1) | | |
| | (GZMB) | | |
| | granzyme A (granzyme 1, cytotoxic T- | GZMA | NM_006144 |
| | lymphocyte-associated serine esterase 3) | | |
| | (GZMA) | | |
| | perforin 1 (pore forming protein) (PRF1) | Perforin | NM_005041 |
| | BCL2-related protein A1 (BCL2A1) | BCL2 | NM_004049 |

### Example 2

The cell-mediated immune profile induced by a recombinant DNA vaccine was assessed in the simian-human immunodeficiency virus (SHIV) and rhesus macaque model. The vaccine strategy included co-immunization of a DNA-based vaccine alone or in combination with a novel optimized plasmid encoding macaque IL-15 (pmacIL-15). Strong induction was observed of vaccine-specific IFN-γ-producing CD8⁺ and CD4⁺ effector T cells in the vaccination groups. Animals were subsequently challenged with 89.6p. The vaccine groups were protected from on-going infection and the IL-15 co-vaccinated group more rapidly controlled infection than the DNA vaccine alone. Lymphocytes isolated from the group co-vaccinated with pmacIL-15 had higher cellular proliferative responses than lymphocytes isolated from the macaques that received SHIV DNA alone. Vaccine antigen activation of lymphocytes was also studied for a series of immunological molecules. While mRNA for IFN-γwas up-regulated following antigen stimulation, the inflammatory molecules IL-8 and MMP-9 were down-regulated. These observed immune profiles are reflective of the ability of the different groups to control SHIV replication. This study demonstrates that an optimized IL-15 immune adjuvant delivered with a DNA vaccine can impact the cellular immune profile in non-human primates and lead to enhanced suppression of viral replication. Importantly, this study indicates that a single read-out such as IFN-γis not the best predictor of viral control.

### Materials and Methods

### Animals

Macaques were housed at the Southern Research Institute in Frederick, Md. These facilities are accredited by the American Association for the Accreditation of Laboratory Animal Care International and meet National Institutes of Health standards as set forth in the Guidelines for Care and Use of Laboratory Animals. Clinical hematology and chemistry studies were performed.

### DNA Plasmids

The pCSIVgag plasmid expresses a 37 kD fragment of the SIV core protein. This rev-independent expression vector and pCSIVpol and pCHIVenv have been optimized for high-level expression as previously described (Nappi F, Scjmeider R, Zolotukhin A, Smulevitch S, Michalowski D, Bear J, Felber B K, Pavlakis G N, (2001) J. Virol 10:4558-4569). The cloning and expression analysis of the macaque IL-15 construct (sequence from Gene Bank, Accession number U19843) was carried out as described in Kutzler et al. (in preparation).

### Immunization Schedule and Sample Collection

Plasmids were manufactured and purified by Puresyn (Malvern, Pa.). Plasmids were greater than 98% supercoiled when formulated. DNA was formulated in 0.15 M citrate solution and 0.25% bupivicaine at a pH of 6.5. The immunization schedule is outlined in Table 1. Groups of 6 cynomologous macaques were immunized three times intramuscularly with either buffer, 2 mg of pSIVgag DNA, or 2 mg of pSIVgag DNA co-injected with 2 mg pmacIL-15. The macaques were then rested 84 weeks prior to performing the second set of immunizations. The second series of immunizations included an increase in dose to 3 mgs of pCSIVgag, pmacIL-15 and incorporated 3 mg of pSIVpol and pHIVenv.

### Peptides

Peptides corresponding to the entire coding region of HIV-lenv and SIVmac239 gag and pol proteins were obtained from the AIDS Reagent Reference Repository (NIH). These 15-mers overlapping by 11 amino acids were resuspended in DMSO at a final concentration of approximately 100 mg/ml and mixed as pools for ELISpot analysis.

### IFN-γELISpot Assay

ELISpot using IFN-γ reagents purchased from MabTech (Sweden) and nitrocellulose plates from Millipore (Billerica, Mass.) as performed previously (Boyer J D, Kumar S, Robinson T, Parkinson R, Wu L, Lewis M, Weiner D B, (2006) J Med Primatol 35:202-209). A positive response is defined as greater than 50 spot forming cells (SFC) per 1 million peripheral blood mononuclear cells (PBMCs) and two times above background. A second set of PBMCs was depleted of CD8⁺ lymphocytes with α-CD8 depletion beads according to manufacturer's protocol (Dynal, Carlsbad, Calif.) before plating cells in triplicate with peptides.

### CFSE Staining for T-Cell Proliferation.

PBMCs were incubated with pre-warmed PBS containing CFSE (5 μM) and incubated for 8 min at 37° C. The cells were washed and incubated with antigens (SIVp27/gag peptide mix) at a concentration of 5 μg/mL for 5 days at 37° C. in 96-well plates. Cultures without gag peptide was used to determine the background proliferative response. Standard surface-staining protocol was followed for CD4⁺ cells using i-human CD4-PE (BD-Pharmingen, San Diego, Calif.) monoclonal antibody. The frequency of CD4⁺ T cells was determined by gating on CD4⁺ T cells. The data were analyzed using the FlowJo program (Ashland, Oreg.).

### RNA Extraction

PBMCs were stimulated from groups 1, 2 and 3 as well as PBMCs isolated from SIV infected Rhesus macaques. The PBMCs from infected macaques served as reference samples. However, while it was expected that SIV infected animals would have an observable immune response to SIVgag, cells from SIV infected macaques were utilized which were being treated with ART to suppress active viral replication thus reducing viral pathogenesis. A total of 5×10⁶ PBMCs were stimulated with SIVgag peptide for 6-12 hr before mRNA was isolated using the RNA-BEE RNA isolation kit (TEL-TEST, Inc., Friendswood, Tex.).

### Gene Expression Analysis

The gene expression patterns of multiple genes were examined by subjecting 25 ng of total RNA from each of the above samples to the GenomeLab™ GeXP Analysis System Multiplex RT-PCR assay (Beckman, Calif.). For each reaction, 3 μl of RNA were mixed with 1.5 μl of 10×DNAse Buffer (Ambion, Tex.) as described earlier (Chen Q, Vansant, G, Oades K, Pickering M, Wei J S, Song Y K, Montforte J, and Khan J, (2007) J Med Diag 9:80-88).

### Infection

Primates were challenged with 300MID by the intravenous (IV) route with SHIV89.6P. (kindly provided by Dr. Norman Letvin, Harvard University) II weeks after the final boost.

### Flow Cytometry

Staining for PD-1 expression was performed using Pacific Blue-conjugated anti-CD3 (clone SP34-2) (BD Pharmingen), PerCP-conjugated anti-CD4 (clone L200) (BD Pharmingen), APC-conjugated anti-CD8 (clone SK1) (BD Biosciences), and Biotin-conjugated anti-PD-1 (R&D Systems cat#BAF1086) for 30 minutes on ice. After washing with PBS, cells were stained using streptavidin-PE (Pharmingen) for 20 minutes on ice. Cells were then washed 3 times in PBS and fixed with 1% paraformaldehyde. Samples were analyzed using a LSRII flow cytometer (BD Biosciences), gating on CD3⁺ lymphocytes.

### SHIV Viral RNA Quantitation

SHIV viral RNA was quantitated using a procedure described by Silvera et al. (Silvera P, Richardson M W, Greenhouse J, Yalley-Ogunro J, Shaw N, Mirchandani J, Kamel Khalili K, Zagury J F, Lewis M G, Rappaport J, (2002) J Virol 76:3800-3809). The assay has a threshold sensitivity of 200 RNA copies/mL of plasma with inter-assay variations averaging 0.5 log₁₀.

### Lymph Node Biopsies and In Situ Hybridization

Formalin-fixed, paraffin-embedded lymph node biopsies were stained for SIV RNA utilizing a method previously described by Hirsch, et al (Hirsch V, Adger-Johnson D, Campbell B, Goldstein S, Brown C, Elkins W R, Montefiori D C. (1997) J Virol 71: 1608-20). The sections were reacted with NBT/BCIP (Vector Laboratories, Ltd., UK) for 10 hr, rinsed with distilled water, counter-stained with nuclear fast red (Sigma), and examined with a Zeiss Z1 microscope.

### Results

The results demonstrated that the cytokine adjuvant pmacIL-15 lead to a unique immunological profile in vivo and significantly impacted viral challenge outcome. Interestingly, while the increase in IFN-γwas not by itself an adequate prediction of the vaccine's ability to control viral challenge; the vaccine appeared to more broadly influence the host immune response. Importantly, infection and vaccination produced unique immune phenotypes.

### IFN-γ Response to SIV239 Gag Following Immunization

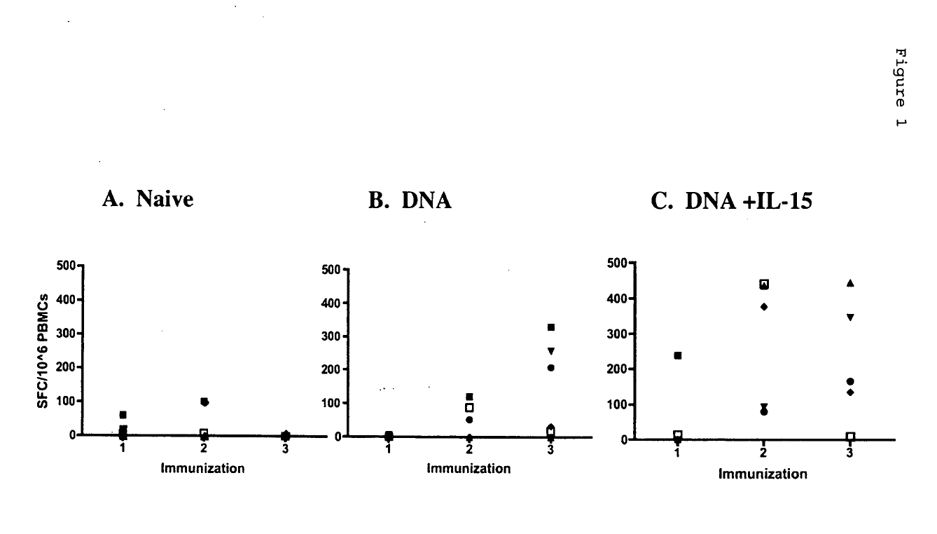

Three groups of cynomologous macaques were immunized by intramuscular injection (Table 2). We assessed the induction of an antigen-specific immune response to SIV gag in all macaques by an IFN-γELISpot assay. Following one immunization there was no measurable immune response in the macaques, which received pCSIVgag (FIG. 1B). However, 1 of 6 with pmacIL-15, (FIG. 1C) did respond. After the second immunization, there was an increase in SIVgag cellular immune responses, with 3 of 6 macaques immunized in Group 2 responding with an average of 135 SFC per 1 million PBMCs. An enhanced gag-specific immune response in Group 3 was also observed in 6 of 6 macaques with an average of 442 SFC per 1 million PBMCs (FIG. 1C). Following the third immunization there appeared to be further boosting in group 2 with little effect in group 3 animals.

TABLE 2
Immunization Schedule

| Immunization | Group 1 | Group 2 | Group 3 |
|---|---|---|---|
| Week 0 | Control | pCSIVgag | pCSIVgag + pmacIL-15 |
| Week 5 | Control | pCSIVgag | pCSIVgag + pmacIL-15 |
| Week 12 | Control | pCSIVgag | pCSIVgag + pmacIL-15 |
| Week 104 | Control | pCSIVgag | pCSIVgag |
| | | pCSIVpol | pCSIVpol + pmacIL-15 |
| | | pCHIVenv | pCHIVenv |
| Week 108 | Control | pCSIVgag | pCSIVgag |
| | | pCSIVpol | pCSIVpol + pmacIL-15 |
| | | pCHIVenv | pCHIVenv |
| Week 112 | Control | pCSIVgag | pCSIVgag |
| | | pCSIVpol | pCSIVpol + pmacIL-15 |
| | | pCHIVenv | pCHIVenv |

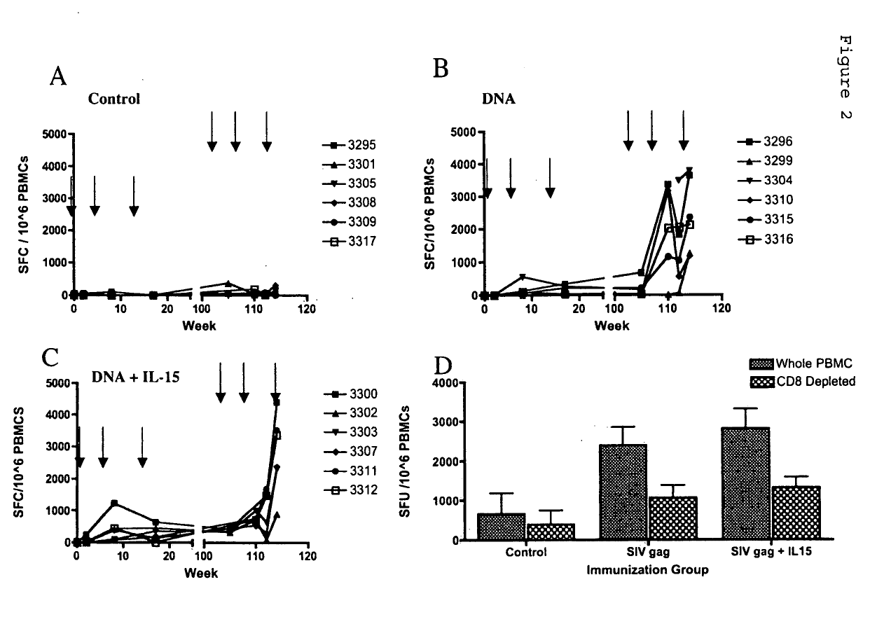

Following a rest period after the third injection macaques were subsequently immunized three times at weeks 104, 108 and 112 with the DNA vaccine that encoded SIV gag. In addition, at this time point STVpol and HIV-lenv plasmids were incorporated. The ELISpot assay was used to monitor the number of SIVgag specific IFN-γ-secreting effector cells (FIG. 2). At the time of the fourth injection, the average number of effector cells in the group immunized with plasmid vaccine alone was 225 SFC/10⁶ PBMCs. Animals that were co-injected with pmacIL-15 resulted in 355 SFC/10⁶ PBMCs.

In a dramatic fashion, the rest period appeared to substantially improve the vaccine induced responses by almost 10 fold. The number of effector cells in Group 2 increased to 2,460 SFC/10⁶ PBMCs following injection 4. Group 3, which received a co-injection of pmacIL-15, also had an increased number of effector cells (2,235 SFC/10⁶ PBMCs). Following injection 5 and 6, the number of effector cells able to secrete IFN-γ in groups 2 (2,389 SFC/10⁶ PBMCs) and 3 (2,389 SFC/10⁶ PBMCs) did not increase significantly (FIG. 2). These data support that substantial immune maturation and expansion of vaccine induced T cells takes place during the extended rest period.

The contribution of CD8⁺ and CD4⁺ T cells to the observed population of cells secreting IFN-γ was evaluated (FIG. 2D). One month post final immunization, isolated PBMCs and PBMCs depleted of CD8⁺T cells were analyzed for the number of cells able to secrete IFN-γ following in vitro stimulation with the gag peptide mix. The animals that were injected with DNA vaccine alone demonstrated a total number of effector cells of 2,389 SFC/10⁶ PBMCs with 1,057 CD4⁺ SFC/10⁶ PBMCS. The animals that were co-immunized with pmacIL-15 had a total of 2,808 SFC/10⁶ PBMCs and 1,316 CD4⁺ SFC/10⁶ PBMCs. Overall the data demonstrates that IFN-γ results were relatively similar between the vaccine groups at the time of viral challenge.

### Control of Viral Replication

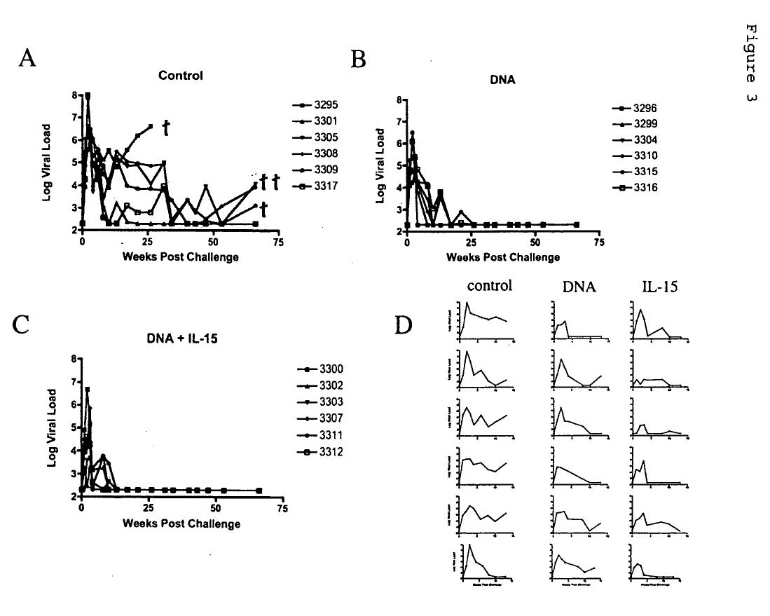

All animals were challenged with SHIV89.6p 11 weeks following the final injection. The average viral loads in the control group at week 2 post challenge or at the peak viral load was 7 logs (FIG. 3A). One macaque was sacrificed at week 25 and 3 at week 60 due to AIDS like symptoms. Animals that received vaccine DNA alone controlled viral load by week 26 (FIG. 3B). Importantly, all animals in the group that received DNA vaccine with pmacIL-15 controlled viral load by week 12 (FIG. 3C). The peak viral loads of group 2 was 5.6 logs. The viral loads for these animals was significantly lower by week 10 when compared to the control group (p=0.020). The average peak viral load for animals that received pmacIL-15 was 3.8 log which was significantly lower than the animals in the control group (p=0.001). Subsequently, the viral loads in this group remained significantly lower than the control group at all time points (p<0.05). In addition, the group of animals that received DNA+pmacIL-15 had a significantly lower viral load at weeks 2, 4 and 6 as compared to the group of animals that received DNA alone.

Lymph node biopsies were taken 57 weeks after challenge. A summary of the results are presented in Table 3. The tissue samples demonstrated that, of the 5 animals that remained alive in the control group, 2 had viral load positive axillary and inguinal lymph nodes. Three of the six animals that received SHIV DNA had either a positive axillary or inguinal lymph node. Only 1 of 6 animals in the group that received IL-15 was demonstrated to be positive for virus.

TABLE 3
Viral Replication in Lymph Nodes

| Control | | | DNA | | | DNA + pmacIL-15 | | |
|---|---|---|---|---|---|---|---|---|
| | Ax | Ing | | Ax | Ing | | Ax | Ing |
| 3295 | t | t | 3296 | + | − | 3300 | + | − |
| 3301 | − | − | 3299 | − | + | 3302 | − | − |
| 3305 | + | + | 3304 | − | + | 3303 | − | − |
| 3308 | + | + | 3310 | − | − | 3307 | − | − |
| 3309 | − | − | 3315 | − | − | 3311 | − | − |
| 3317 | NT | − | 3316 | − | − | 3312 | NT | − |

t = terminated prior to biopsy, Ax = axilary lymph node, Ing = Inguinal lymph node, + = positive, − = negative, NT = no tissue

### IFN-γ-Producing Cells Following Challenge

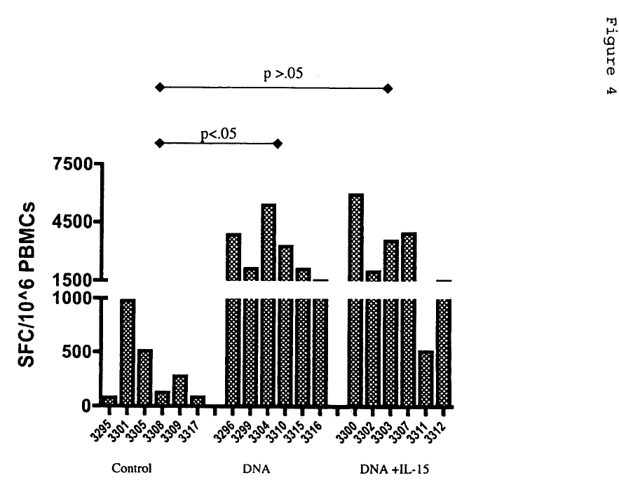

Samples were obtained and studied 12 weeks post challenge and assessed for the number of cells able to secrete IFN-γfollowing in vitro stimulation with SIVgag, FIG. 3. The cellular immune responses in the control group, while above detectable limits, at no time point reached the same level of responses generated in the vaccine groups (FIG. 4). However animal 3301, in the control group, was able to control viral replication. This animal reached a level of 1000 SFC per 1 million PBMCs. Yet, the group that was immunized with DNA vaccine alone had a significantly higher level of IFN-γ compared to the control group at week 12 post challenge. Interestingly, the low secondary response of animal 3311 resulted in a lack of a significant difference between the control group and the animals that received DNA combined with IL-15. This data demonstrates that as virus replicates, the T cell immune response to SIV antigens induced by the vaccine is modulated by antigen encountered by immune cells during viral replication. We next sought to examine additional markers to gain additional insight into the basis for immune control by these vaccines.

### PD-1 Expression

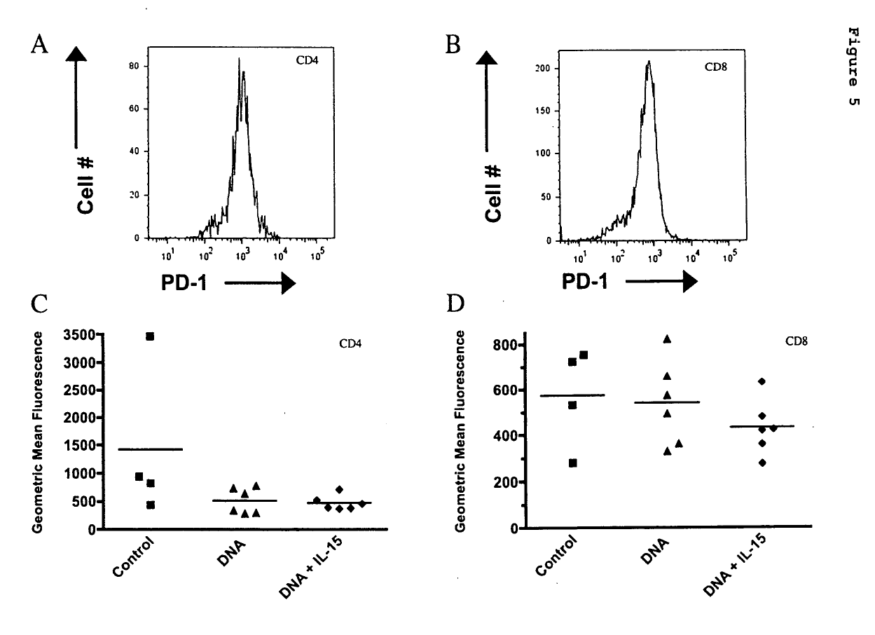

The level of PD-1 expression was assessed following viral challenge. The mean fluorescent expression of PD-1 on CD4+ and CD8+ T cells was higher in macaques that were unvaccinated and had on-going viral replication. The lower level of PD-1 expression in vaccinated macaques is a further indication that viral replication in these animals is suppressed, thereby preserving the healthy immune systems of these animals (FIG. 5).

### Proliferation of T Cells by DNA and IL-15 Co-Injected Groups.

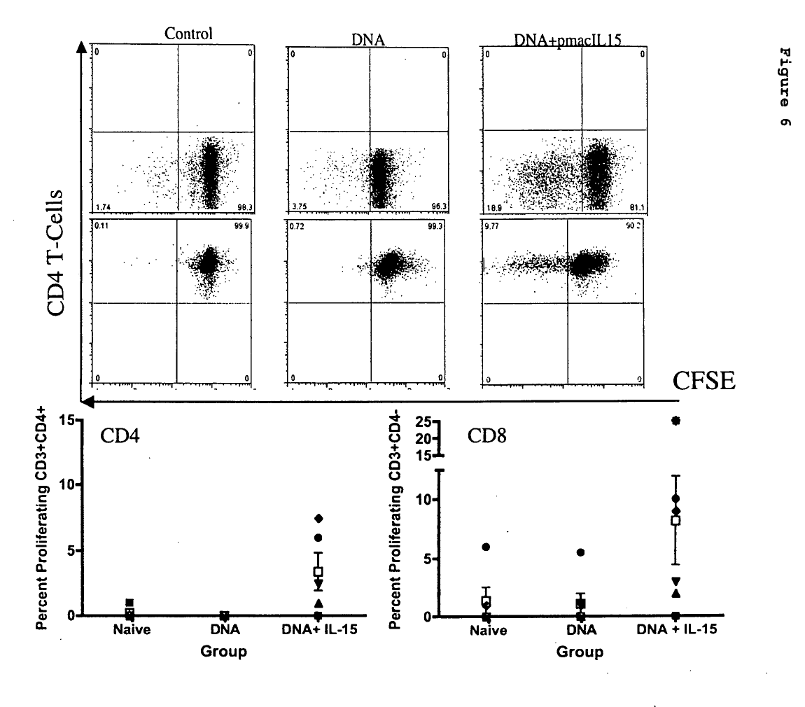

Lymphocytes that are fully differentiated are capable of proliferating following in vitro antigen stimulation. The ability of the vaccine-specific CD8⁺ and CD4⁺ effector cells to proliferate in vitro following SIVgag antigen stimulation was investigated. PBMCs were isolated from all macaques 2 weeks following the final immunization. Cells were incubated with CFSE, washed and stimulated with SIVgag antigen for 5 days. The data obtained from CFSE proliferation study demonstrated no proliferation in the control group, and little to no proliferative capacity for the lymphocytes isolated from macaques immunized with DNA vaccine. The proliferative responses were dramatically improved in pmacIL-15 co-immunized animal groups (FIG. 6). An average of 3% gag-specific CD4⁺ T-cell and 8% of the CD8⁺ cells were proliferating in the pmacIL-15 co-vaccinated animals.

### Inflammatory Panel of Cytokines

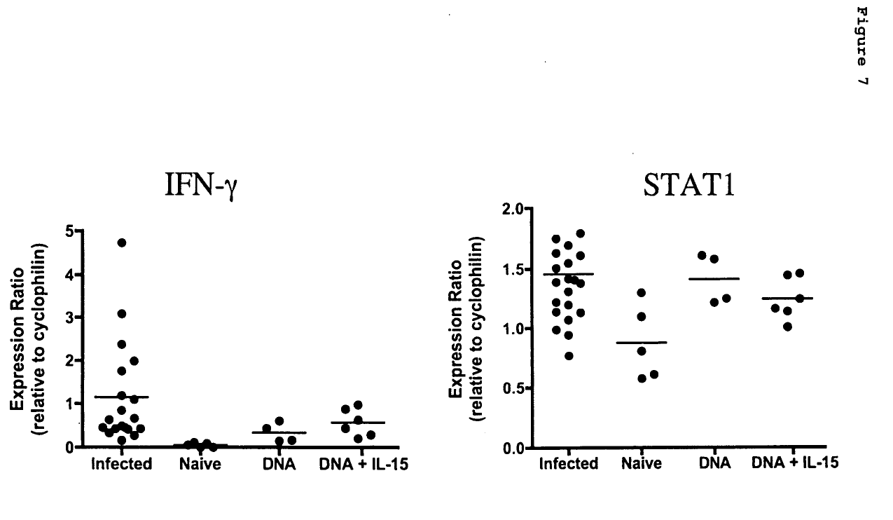

In order to further understand the immunological profiles induced by these DNA vaccines, PBMCs taken after the 6th and final vaccination were stimulated in vitro for 6-12 hours with SIVgag antigen. Following in vitro stimulation, RNA was isolated. In addition to our naive controls and the two vaccine groups, PBMCs from SIV infected macaques were isolated and stimulated in vitro in an analogous manner to the vaccine group. Antigen specific expression levels of a number of genes were altered as a result of SIV DNA vaccination and are presented. The genes for IFN-γ and STAT1 (FIG. 7) are clearly upregulated in PBMCs isolated from vaccinated macaques and stimulated with SIVgag. There was no expression of IFN-γ following antigen stimulation of PBMCs in the control group.

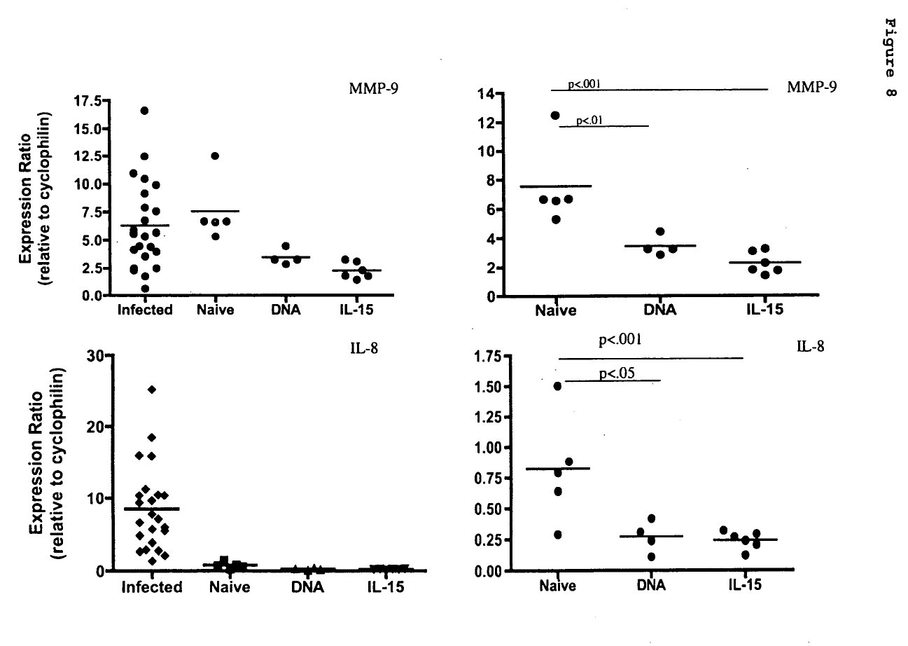

It was further observed that MMP9 and IL-8 (FIG. 8) are down modulated in antigen specific cells in the vaccine groups as compared to naive cells. However, MMP-9 and IL-8 gene expression is high in SIV infected virally suppressed macaques when the PBMCs were stimulated with SIVgag antigen. Furthermore, IL-8 gene expression in PBMCs of infected macaques was higher than PBMCs isolated from naive macaques with SIVgag antigen.

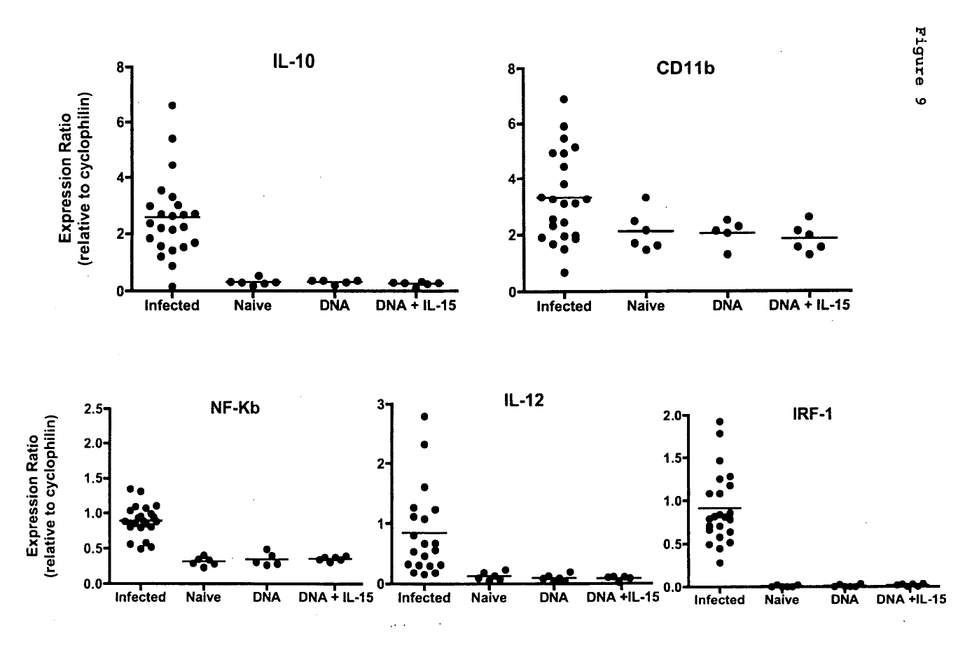

FIG. 9 illustrates that while several genes do not vary between naive and vaccinated animals, there is a clear increase in specific immune related gene expression in SIV infected macaques. Specifically, gene expression for IL-10, CD11b, NFκB, IL-12 and IRF-1 are increased above background levels or those observed in a naive animals. NFκB and IRF-1 are particularly interesting as HIV requires these transcription factors for its efficient intracellular replication. Antigen specific cells that are not upregulated in NFκB and IRF-1 expression may reduce the number of target cells for HIV-1 replication. These data suggests that an effective T cell vaccine can drive immune expansion in a manner that does not provide necessary molecules for efficient pathogen replication.

### Discussion

The experiments demonstrated significant protection against SHIV89.6p replication and pathogenesis in macaques co-immunized with SHIV DNA and a plasmid IL-15 adjuvant. Both vaccine groups could control viral replication however there were important differences. Co-immunization with pmacIL-15 lead to an increased ability to rapidly suppress viral replication and control. The group that was vaccinated with DNA alone also was able to control viral replication, however the viral peak was higher in and control of viral replication in all animals took to week 25. Over the course of immunization, we noted that IL-15 plasmid did not appear to dramatically increase the magnitude of the IFN-γ producing cellular immune response. However, these animals did have higher proliferative responses, with a higher ratio of CD8⁺ T-cell proliferation compared to CD4⁺ T-cell proliferation.

15 genes associated with induction of a cellular immune response were assessed. The level of IFN-γ gene expression increased when cells were stimulated with SIVgag concurrently with increased gene expression of STAT1 a signal transducer activated by interferon. The down modulation of the genes encoding IL-8 and matrix metalloproteinase 9 (MMP-9), two molecules associated with the proinflammatory state and establishment of chronic infection was of high interest. IL-8 is a chemokine produced by monocytes, macrophages, fibroblasts and endothelial cells, and is produced during the inflammatory response to signal neutrophils. The matrix metalloproteinases (MMPs) are a family of extracellular endopeptidases that selectively degrade components of the extracellular matrixes. A decreased level of MMP-9 gene expression was observed when cells from vaccinated macaques were stimulated with antigen as compared to naive animals stimulated with SIVgag antigen. As a state of chronic inflammation appears to be associated with the establishment of HIV/SIV infection, the change in MMP-9 and IL-8 expression appears very interesting.

The transcriptional regulators of the NFκB/IκB family promote the expression of well over 100 target genes, the majority of which participate in the host immune response. Persistent activation of the NFκB pathway can lead to oncogenic transformation. It is quite clear from the observations of mRNA expression that perhaps the correlates of protection are not as yet presenting a clear picture.

This demonstrates that DNA vaccination with pmacIL-15 immunoadjuvant can contribute to enhanced immune profiles in a novel defined fashion and control of SHIV89.6P infection. The number of effector cells able to secrete IFN-γis most likely not the sole correlate of protection. Further examination of such defined and adjuvanted DNA vaccines in challenge settings appear to be a useful tool for probing correlates of pathogen immunity and may provide interesting immune phenotypes for clinical study.

The disclosures of all US patents and applications specifically identified herein are expressly incorporated herein by reference. Particular features of the invention are emphasized in the claims which follow.

What is claimed:

1. A method for evaluating a vaccine in a subject comprising determining in the subject the activity of at least two genes selected from the group consisting of genes associated with the proimflammatory state and genes associated with induction of a cellular immune response in the subject, wherein a collective result of the activity of at least two genes is indicative of the efficacy of the vaccine.

2. The method of claim 1, wherein determining the activity of at least two genes comprises determining gene transcription level, gene translation level, protein activation level, or a combination thereof.

3. The method of claim 1, wherein genes associated with the proimflammatory state include IL8, MMP9, IL1, IL2, IL4, IL6, IL10, IL12B, IL18, TNF, IFN-γ, FGF1, CD63, CTGF, CCL1, NFkB1, STAT1, CSF1, MCP, MCP-1RB, IRF-1, and HSGPCRIN8.

4. The method of claim 1, wherein genes associated with induction of a cellular immune response include IFN-γ, STAT1, IL10, CD11b, NFkb, IL12, IRF-1, CYP1B1, ICAM1, CYP1A1, TP53, IL1B, IL15, CD14, LTA, CD4, NOS2A, CSF2, CYP1A2, PTGS2, CASP9, CD8A, MYC, PTPRC, NFkB1, CASP3, IL23A, and SCIN.

5. The method of claim 1, wherein a decrease in the activity of a gene associated with the proimflammatory state is indicative of the efficacy of the vaccine.

6. The method of claim 1, wherein an increase in the activity of a gene associated with the induction of a cellular immune response is indicative of the efficacy of the vaccine.

7. The method of claim 1, wherein a decrease in the activity of a gene associated with the proimflammatory state and an increase in the activity of a gene associated with the induction of a cellular immune response is indicative of the efficacy of the vaccine.

8. The method of claim 1, wherein a decrease in the activity of IL8, MMP9, IL1, IL2, IL4, IL6, IL10, IL12B, IL18, TNF, IFN-γ, FGF1, CD63, CTGF, CCL1, NFkB1, STAT1, CSF1, MCP, MCP-1RB, IRF-1, and HSGPCRIN8 is indicative of the efficacy of the vaccine.

9. The method of claim 1, wherein an increase in the activity of IFN-γ, STAT1, IL-10, CD11b, NFkb, IL-12, IRF-1, CYP1B1, ICAM1, CYP1A1, TP53, IL1B, IL15, CD14, LTA, CD4, NOS2A, CSF2, CYP1A2, PTGS2, CASP9, CD8A, MYC, PTPRC, NFkB1, CASP3, IL23A, and SCIN is indicative of the efficacy of the vaccine.

10. The method of claim 1 comprising determining the activity of at least three, four, or five genes.

11. The method of claim 1, further comprising vaccinating the subject with the vaccine prior to said evaluating.

12. The method of claim 1, further comprising obtaining a sample from said subject prior to said evaluating.

13. The method of claim 12, wherein the sample is a blood sample.

14. A combination of tests useful for predicting whether a vaccine is effective comprising
a first test for the activity of a first gene and
a second test for the activity of a second gene,
wherein the first gene and the second gene are selected from the group consisting of genes associated with the proimflammatory state and genes associated with induction of a cellular immune response.

15. The combination of tests of claim 14, wherein the first gene or the second gene is selected from the group consisting of IL8, MMP9, IFN-γ, STAT1, IL10, CD11b, NFkB1, IRF-1, IL6, TGFbeta, IL4, CSF1, CCR2, GZMA, GZMB, TNFalpha, FGF1, PD1, CCL4, Perforin, MCP1, IL18, and CXCR3.

16. The combination of tests of claim 14, wherein the activity of a first gene and a second gene comprises gene transcription level, gene translation level, or protein activation level, or a combination thereof.

17. The combination of tests of claim 14 further comprising a third test for the activity of a third gene, wherein the third gene is selected from the group consisting of genes associated with the proimflammatory state and genes associated with induction of a cellular immune response.

18. The combination of tests of claim 14 further comprising a third test for the proliferation of T-cells.

19. A method of providing useful information for evaluating whether a vaccine is effective comprising
determining the activity of a first set of genes and
providing the activity of the first set of genes to an entity that analyzes the activity and provides an evaluation of the vaccine,
wherein the first set of genes comprises at least two genes selected from the group consisting of genes associated with proimflammatory state and genes associated with induction of a cellular immune response.

20. The method of claim 19, wherein the first set of genes are selected from the group consisting of IL8, MMP9, IFN-γ, STAT1, IL10, CD11b, NFkB1, IRF-1, IL6, TGFbeta, IL4, CSF1, CCR2, GZMA, GZMB, TNFalpha, FGF1, PD1, CCL4, Perforin, MCP1, IL18, and CXCR3.

21. The method of claim 19, wherein the activity of the first set of genes are provided in a format compatible with a computer algorithm used by the entity for analysis.

22. The method of claim 19, wherein the first set of genes are at least three genes selected from the group consisting of IL8, MMP9, IFN-γ, STAT1, IL10, CD11b, NFkB1, IRF-1, IL6, TGFbeta, IL4, CSF1, CCR2, GZMA, GZMB, TNFalpha, FGF1, PD1, CCL4, Perforin, MCP1, IL18, and CXCR3.

23. The method of claim 19, wherein the first set of genes are at least four genes selected from the group consisting of IL8, MMP9, IFN-γ, STAT1, IL10, CD11b, NFkB1, IRF-1, IL6, TGFbeta, IL4, CSF1, CCR2, GZMA, GZMB, TNFalpha, FGF1, PD1, CCL4, Perforin, MCP1, IL18, and CXCR3.

24. A collection of results useful for evaluating whether a vaccine is effective comprising values for the activity of a first set of genes selected from the group consisting of genes associated with proinflammatory state and genes associated with induction of a cellular immune response, wherein the first set of genes comprises at least two genes.

25. The collection of results of claim 24, wherein the first set of genes are selected from the group consisting of IL8, MMP9, IFN-γ, STAT1, IL10, CD11b, NFkB1, IRF-1, IL6, TGFbeta, IL4, CSF1, CCR2, GZMA, GZMB, TNFalpha, FGF1, PD1, CCL4, Perforin, MCP1, IL18, and CXCR3.

26. The collection of results of claim 24, wherein the first set of genes comprises at least three, four or five genes.

27. The collection of results of claim 24, wherein said values are in a format compatible with a computer algorithm for analyzing the results and providing an evaluation of the vaccine.

28. A collection of nucleotides useful for determining the activity of a set of genes selected from the group consisting of genes associated with proimflammatory state and genes associated with induction of a cellular immune response, wherein the set of genes comprises at least two genes.

29. The collection of claim 28, wherein the nucleotides can be useful for determining the activity of a set of genes in two or more species.

30. The collection of claim 28, wherein the nucleotides are probes for determining the gene expression level or gene translation level of the set of genes.

31. The collection of claim 28, wherein the nucleotides are probes for determining the gene expression level or gene translation level of the set of genes and wherein the nucleotides are provided on a solid support.

32. The collection of claim 28, wherein the nucleotides are primers for determining the gene expression level or gene translation level of the set of genes.

33. A reaction mixture comprising primers or probes useful for determining the activity of a set of genes selected from the group consisting of genes associated with proimflammatory state and genes associated with induction of a cellular immune response, wherein the set of genes comprises at least two genes.

34. The reaction mixture of claim 33 comprising amplified products corresponding to the set of genes.

35. The reaction mixture of claim 33 comprising primers and probes useful for determining the activity of the set of genes.

36. The reaction mixture of claim 33, wherein the set of genes comprises at least three, four, or five genes.

37. The reaction mixture of claim 33, wherein the set of genes are selected from the group consisting of IL8, MMP9, IFN-γ, STAT1, IL10, CD11b, NFkB1, IRF-1, IL6, TGFbeta, IL4, CSF1, CCR2, GZMA, GZMB, TNFalpha, FGF1, PD1, CCL4, Perforin, MCP1, IL18, and CXCR3.
